(19) United States
(12) Patent Application Publication
(10) Pub. No.: US 2008/0317751 A1
(43) Pub. Date: Dec. 25, 2008

(54) Idiotype Vaccination with Bispecific and Multispecific Immunoglobulin Molecules

(76) Inventor: Andrew William Heath, Sheffield (GB)

Correspondence Address: KLARQUIST SPARKMAN, LLP, 121 SW SALMON STREET, SUITE 1600, PORTLAND, OR, 97204, US

(21) Appl. No.: 11/917,568

(22) PCT Filed: Jun. 15, 2006
(86) PCT No.: PCT/GB2006/002193

§ 371 Date: Jun. 9, 2008

(30) Foreign Application Priority Data
Jun. 16, 2005 (GB) 0512225.4

(51) Int. Cl. A61K 39/395; C07K 16/18; C12N 15/11; C12N 15/00; A61K 31/7088; A61P 37/00; C12N 15/87; C12N 5/06; C12N 1/19; C12N 1/20

(52) U.S. Cl. 424/136.1; 530/387.3; 536/23.53; 435/320.1; 435/325; 435/254.2; 435/252.3; 514/44; 435/451; 435/346

(57) ABSTRACT

The present application relates to immunoglobulin polypeptides comprising at least two immunoglobulin complementarity determining regions, one that binds to an immune receptor and the other which comprises an idiotype to which an immune response is desired.

The invention relates to a molecule containing at least two immunoglobulin complementarity determining regions, one of which binds to an immune receptor polypeptide, and the other of which is an antigen against which an immune response is desired.

An adjuvant is a substance or procedure which augments specific immune responses to antigens by modulating the activity of immune cells. Examples of adjuvants include Freunds adjuvant, muramyl dipeptides, liposomes. An adjuvant is therefore an immunomodulator and distinct from a “carrier” which is an immunogenic molecule which, when bound to a second molecule augments immune responses to the latter largely through the provision of additional T cell epitopes. One of the most important developments in the history of medicine is the advent of vaccines which are used to protect against a wide variety of infectious and non-infectious diseases. Many vaccines are produced by inactivated or attenuated pathogens which are injected into an individual. Many modern vaccines are made from protective antigens of the pathogen. These latter vaccines are known as ‘subunit vaccines’. Although subunit vaccines tend to avoid the side effects of killed or attenuated pathogen vaccines, their ‘pure’ status has separated from the ‘danger signals’ that are often associated with whole organism vaccines, and subunit vaccines do not always have adequate immunogenicity. Many candidate subunit vaccines have failed in clinical trials in recent years that might otherwise have succeeded were a suitable adjuvant available to enhance the immune response to the purified antigen.

Antibodies or immunoglobulins are protein molecules which have specificity for foreign molecules (antigens). Immunoglobulins (Ig) are a class of structurally related proteins consisting of two pairs of polypeptide chains, one pair of light (L) (low molecular weight) chain (κ or λ), and one pair of heavy (H) chains (γ, α, μ, δ and ε), all four linked together by disulphide bonds. Both H and L chains have regions that contribute to the binding of antigen and that are highly variable from one Ig molecule to another. The L chains consist of two domains. The carboxy-terminal domain is essentially identical among L chains of a given type and is referred to as the “constant” (C) region. The amino terminal domain varies from L chain to L chain and contributes to the binding site of the antibody.

Because of its variability, it is referred to as the “variable” (V) region. The H chains of Ig molecules are of several classes (α, μ, σ, α, and γ of which there are several sub-classes). An assembled Ig molecule consisting of one or more units of two identical H and L chains derives its name from the H chain that it possesses. Thus, there are five Ig isotypes: IgA (with 2 subclasses, IgA1 and IgA2), IgM, IgD, IgE and IgG (with four sub-classes based on the differences in the H chains, i.e., IgG1, IgG2, IgG3 and IgG4). Further detail regarding antibody structure and their various functions can be found in, Using Antibodies: A laboratory manual, Cold Spring Harbour Laboratory Press.

The region of an antibody that determines the binding specificity of the antibody for its antigen is referred to as the complementarity determining region (CDR) and is also referred to as the “hypervariable region” or the “idiotype”. Because the antigen binding regions of antibodies are made up of amino acid sequences derived at random they are unique to one clone or a small number of clones of B cells. These unique peptide sequences are therefore antigenic in their own right, and in combination serve to make up the antibody molecule's unique idiotype. As an antigen is made up of a number of epitopes, so also an idiotype is made up of a number of “idiotopes”. Immunisation with purified immunoglobulin of a particular idiotype (often a monoclonal antibody) can generate antibody responses against that idiotype. In turn, these antibodies will induce their own anti-idiotype responses, and so on. This process was first hypothesised by Jerne et al called the network theory (Jerne, Niels K. (1974) “Towards a network theory of the immune system,” Annals of Institute Pasteur/Immunology (Paris) 125C: 373-389).

There are two systems that use immunoglobulins as vaccine antigens, with the aim of inducing an immune response against the immunoglobulin. In both systems it is the hypervariable region or idiotype of the antibody that is used to provoke an immune response. In both cases the idiotype of the antibody is used to generate an anti-idiotype response (anti-Id). In the first case, the idiotype of the antibody is the actual target of the immune effector response. For instance B cell lymphomas and leukemias are generally derived from a single clone of B cells and thus may express on their cell surface an immunoglobulin which is unique or almost unique to the tumour (1) The generation of an immune response against this immunoglobulin idiotype is the desired effect of vaccination, which may aid in clearance of the tumour cells (2). The anti-idiotype response generated can consist of both antibody and T cell mediated responses. Immunoglobulin idiotypes can thus be one of the best examples of a tumour specific antigen. There may be other cases in which a particular antibody idiotype is associated with disease, such as for instance in autoimmune disease where the response to the autoantigen is mediated predominantly by antibodies of a particular idiotype, or a small number of idiotypes (e.g. Routsias et al. Molecular Medicine 8.6 (2002) 293-305)

The second system uses a so called “internal image anti-idiotype antibody” to generate a response which cross-reacts with an antigen, which may be a tumour antigen or an antigen from a pathogen or another source which for one reason or another is difficult to purify or is poorly immunogenic when administered directly. The system of nomenclature for idiotype (Id)anti-idiotype (anti-Id) interactions is complex, and is described in more detail elsewhere (Thanavala, Trends in Biotechnology 7, 62-66, 1989), Briefly however, an antibody such as a monoclonal antibody able to bind an antigen, such as a ganglioside, would be termed Ab1. If this Ab1 antibody were used for immunisation, it would generate anti-Idiotype antibodies against itself, known as Ab2. There are two easily distinguishable kinds of Ab2 antibodies, those which will inhibit the binding of the Ab1 to its antigen, known as Ab2β, and those which will not, known as Ab2α. Some of the Ab2β antibodies, because they are binding Ab1 within the antigen binding site, will have a structure very similar to the antigen itself, so they are known as “internal image anti-Id antibodies”. Because they look like antigen, if they are used as vaccine antigens they will generate an immune response against the target antigen itself (for instance the ganglioside). In fact internal image anti-idiotype vaccines can generate T cell responses against the original polypeptide antigen, as well as antibody responses against the original antigen.

Idiotype based vaccines, including anti-idiotype vaccines as described above, are known in the art. However, these vaccines have associated problems. Firstly, for lymphoma patients the vaccines must be individually produced as the idiotype is likely to be unique to that individual's tumour; and it can take up to several months to formulate the vaccine which often involves producing hybridomas secreting the desired idiotype, purifying the immunoglobulin and then conjugating to a protein carrier like KLH (2, 3). Secondly, human immunoglobulins are inherently poorly immunogenic in humans, so despite conjugation to a carrier to augment the immune response to the idiotype, anti-Id antibody responses tend to be weak (in fact a large proportion of the response to the conjugates is directed at the highly immunogenic carrier protein). Thirdly, it has been shown in both mice and humans that both CD4+ T cells and CD8+ CTL responses against the idiotype protein may be important in mediating the therapeutic response (4-6), and conjugation to a carrier such as KLH is not the most efficient means of generating CTL responses.

In co-pending applications WO04/052396, EP03734751.5 and WO04/064864 we describe various polypeptides and polypeptide complexes which have potent adjuvant activity. The polypeptides/polypeptide complexes comprise the antibody binding region of an antibody that binds the immune receptors CD28 and CD40 and conjugated to the antibody an antigen to which an immune response is desired. The conjugation of the antigen to a CD28 or CD40 antibody greatly augments the immune response to the associated antigen. Importantly with respect to idiotype vaccination, CD40 antibodies induce a very strong response against themselves (7). When rat anti-mouse CD40 is used to immunise mice, the anti-rat IgG response induced is around 1000-fold stronger than the response against an irrelevant isotype matched rat antibody. CD40 antibody as an adjuvant has also been shown to very strongly enhance T cell responses against both conjugated antigens, and against rat IgG2a (10).

We describe a novel approach to idiotype vaccination which would result in a much more immunogenic vaccine and which addresses some of the problems associated with anti-idiotype vaccines. For example, idiotype vaccines against lymphomas produced using methods described herein may be more highly immunogenic, may be produced more quickly and may produce stronger idiotype specific T cell responses than current methods.

According to an aspect of the invention there is provided an immunoglobulin molecule wherein said molecule comprises a first part that binds to an immune cell receptor polypeptide and a second part that comprises an idiotype to which an immune response is desired.

In a preferred embodiment of the invention said first part comprises at least one heavy chain or at least one light chain immunoglobulin variable region. Preferably said second part comprises at least one heavy chain immunoglobulin heavy chain or at least one immunoglobulin light chain.

According to a further aspect of the invention there is provided a multivalent immunoglobulin polypeptide wherein said polypeptide comprises a first part that includes more than one variable region that binds to an immune cell receptor and a second part that includes more than one idiotype to which an immune response is desired.

In a preferred embodiment of the invention said multivalent immunoglobulin comprises heavy and/or light variable regions from immunoglobulin class IgM or IgA.

Various fragments of immunoglobulin or antibodies are known in the art, i.e., Fab, Fab₂, F(ab′)₂, Fv, Fc, Fd, scFvs, etc. A Fab fragment is a multimeric protein consisting of the immunologically active portions of an immunoglobulin heavy chain variable region and an immunoglobulin light chain variable region, covalently coupled together and capable of specifically binding to an antigen. Fab fragments are generated via proteolytic cleavage of an intact immunoglobulin molecule. A Fab₂ fragment comprises two joined Fab fragments. When these two fragments are joined by the immunoglobulin hinge region, a F(ab′)₂ fragment results. An Fv fragment is multimeric protein consisting of the immunologically active portions of an immunoglobulin heavy chain variable region and an immunoglobulin light chain variable region covalently coupled together and capable of specifically binding to an antigen. A fragment could also be a single chain polypeptide containing only one light chain variable region, or a fragment thereof that contains the three CDRs of the light chain variable region, without an associated heavy chain variable region, or a fragment thereof containing the three CDRs of the heavy chain variable region, without an associated light chain moiety; and multi specific antibodies formed from antibody fragments, this has for example been described in U.S. Pat. No. 6,248,516. Fv fragments or single region fragments are typically generated by expression in host cell lines of the relevant identified regions. These and other immunoglobulin or antibody fragments are within the scope of the invention and are described in standard immunology textbooks such as Paul, Fundamental Immunology or Janeway et al. Immunobiology.

In a preferred embodiment of the invention said first part comprises a heavy or light chain variable region from an immunoglobulin selected from the group consisting of: an IgM, an IgD, an IgG, an IgA or an IgE.

In a preferred embodiment of the invention said IgG is selected from the group consisting of: IgG1, IgG2, IgG3 and IgG4.

In a preferred embodiment of the invention said fragment is selected from the group consisting of: an Fv fragment, a Fab fragment, a F(ab′)₂ fragment, a F(ab)₂ fragment, a scFvs fragment, a single chain antibody fragment, a single domain antibody.

It is possible to create single variable regions, so called single chain antibody variable region fragments (scFvs). If a hybridoma exists for a specific monoclonal antibody it is well within the knowledge of the skilled person to isolate scFvs from mRNA extracted from said hybridoma via RT PCR. Alternatively, phage display screening can be undertaken to identify clones expressing scFvs.

Alternatively, the fragments are “domain antibody fragments”. Domain antibodies are the smallest binding part of an antibody (approximately 13 kDa). Examples of this technology is disclosed in U.S. Pat. No. 6,248,516, U.S. Pat. No. 6,291,158, U.S. Pat. No. 6,127,197 and EP0368684 which are all incorporated by reference.

In a preferred embodiment of the invention said first part consists of a complementarity determining region of an immunoglobulin.

In a preferred embodiment of the invention said second part comprises at least one idiotope against which an immune response is desired.

In a further preferred embodiment of the invention said first part is crosslinked or conjugated to said second part.

In another embodiment of the invention said first part and said second part are linked to each other by cross-linking molecule.

In a further embodiment of the invention said first part and said second part are associated in a vehicle such as a liposome or a microparticle, or are cross-linked to a liposome or microparticle.

In another embodiment of the invention said first part and said second part are co-emulsified in an oil-based emulsion or co-precipitated onto a carrier material.

In a preferred embodiment of the invention said polypeptide is a fusion protein wherein said first and second parts are in frame translational fusions.

In a preferred embodiment of the invention said first part binds a member of the tumour necrosis factor receptor superfamily,

In a further preferred embodiment of the invention said first part binds the immune receptor CD40.

In a further preferred embodiment of the invention said first part binds OX40, Fas (CD95), BAFF receptor, BCMA, TACI, APRIL receptor, CD27, CD134, or CD137

In a preferred embodiment of the invention said first part binds a member of the immunoglobulin superfamily other than immunoglobulin.

In a further preferred embodiment of the invention said first part binds the immune receptor CD28.

In another preferred embodiment of the invention said first part binds CD152, CD80 or CD86 or ICOS.

In another embodiment of the invention said first part binds a member of the TNF superfamily

In a further embodiment said first part binds CD154, Fas ligand, APRIL or TRAIL.

In another preferred embodiment of the invention said first part binds a dendritic cell surface antigen, such as DEC 205, CD11c or DC-SIGN.

In a further preferred embodiment of the invention said first part binds a complement receptor such as CD21.

In another embodiment of the invention said first part binds a cytokine receptor or a chemokine receptor.

In another preferred embodiment of the invention said first part binds an adhesion molecule, such as an integrin.

In a preferred embodiment of the invention said first part is derived from monoclonal antibody or binding fragment thereof. Preferably said monoclonal antibody is a humanised or chimeric antibody.

A chimeric antibody is produced by recombinant methods to contain the variable region of an antibody with an invariant or constant region of a human antibody.

A humanised antibody is produced by recombinant methods to combine the complementarity determining regions (CDRs) of an antibody with both the constant (C) regions and the framework regions from the variable (V) regions of a human antibody.

Chimeric antibodies are recombinant antibodies in which all of the V-regions of a mouse or rat antibody are combined with human antibody C-regions. Humanised antibodies are recombinant hybrid antibodies which fuse the complimentarily determining regions from a rodent antibody V-region with the framework regions from the human antibody V-regions. The C-regions from the human antibody are also used. The complimentarily determining regions (CDRs) are the regions within the N-terminal domain of both the heavy and light chain of the antibody to where the majority of the variation of the V-region is restricted. These regions form loops at the surface of the antibody molecule. These loops provide the binding surface between the antibody and antigen.

Antibodies from non-human animals provoke an immune response to the foreign antibody and its removal from the circulation. Both chimeric and humanised antibodies have reduced antigenicity when injected to a human subject because there is a reduced amount of rodent (i.e. foreign) antibody within the recombinant hybrid antibody, while the human antibody regions do not elicit an immune response. This results in a weaker immune response and a decrease in the clearance of the antibody. This is clearly desirable when using therapeutic antibodies in the treatment of human diseases. Humanised antibodies are designed to have less “foreign” antibody regions and are therefore thought to be less immunogenic than chimeric antibodies.

In a further preferred embodiment of the invention, if part 2 comprises an internal image anti-idiotype antibody generated in a non-human animal, this part will be chimeric, and will contain human constant regions.

In a preferred embodiment of the invention said immunoglobulin is provided with a sequence tag to facilitate isolation/purification.

A number of such sequence tags are known to those skilled in the art, or can easily be devised. They might include, by way of example only, polyhistidine sequences to allow purification on Nickel or cobalt columns, or antibody epitopes such as the Flag™ sequence to facilitate purification by antibody affinity.

According to a further aspect of the invention there is provided a nucleic acid molecule comprising a nucleic acid sequence that encodes an immunoglobulin according to the invention.

According to an aspect of the invention there is provided a vector, preferably an expression vector that comprises a nucleic acid molecule according to the invention.

In a preferred embodiment of the invention said vector is a plasmid, viral based vector, phage or phagemid.

According to a further aspect of the invention there is provided a cell transformed or transfected with a nucleic acid or vector according to the invention.

In a preferred embodiment of the invention said cell is a eukaryotic cell.

In an alternative embodiment of the invention said cell is a prokaryotic cell; preferably a bacterial cell.

According to a further aspect of the invention there is provided a pharmaceutical composition comprising an immunoglobulin according to the invention.

According to an alternative aspect of the invention there is provided a pharmaceutical composition comprising a nucleic acid molecule or vector according to the invention.

When administered the compositions of the present invention are administered in pharmaceutically acceptable preparations. Such preparations may routinely contain pharmaceutically acceptable concentrations of salt, buffering agents, preservatives, compatible carriers, supplementary immune potentiating agents such as adjuvants and cytokines and optionally other therapeutic agents, such as chemotherapeutic agents which can be administered separately from the polypeptides/nucleic acids of the invention or in a combined preparation if a combination is compatible.

The therapeutics of the invention can be administered by any conventional route, including injection or by gradual infusion over time. The administration may, for example, be oral, intravenous, intraperitoneal, intramuscular, intracavity, subcutaneous, or transdermal.

The compositions of the invention are administered in effective amounts. An “effective amount” is that amount of a composition that alone, or together with further doses, produces the desired response. In the case of treating a particular disease, such as cancer, the desired response is inhibiting the progression of the disease. This may involve only slowing the progression of the disease temporarily, although more preferably, it involves halting the progression of the disease permanently. This can be monitored by routine methods.

Such amounts will depend, of course, on the particular condition being treated, the severity of the condition, the individual patient parameters including age, physical condition, size and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

The pharmaceutical compositions used in the foregoing methods preferably are sterile and contain an effective amount of nucleic acid or immunoglobulin for producing the desired response in a unit of weight or volume suitable for administration to a patient. The response can, for example, be measured by determining regression of a tumour, decrease of disease symptoms, modulation of apoptosis, etc.

The doses of nucleic acid or immunoglobulin administered to a subject can be chosen in accordance with different parameters, in particular in accordance with the mode of administration used and the state of the subject. Other factors include the desired period of treatment. In the event that a response in a subject is insufficient at the initial doses applied, higher doses (or effectively higher doses by a different, more localized delivery route) may be employed to the extent that patient tolerance permits.

In general, doses of nucleic acids of between 1 ng and 0.1 mg generally will be formulated and administered according to standard procedures. Other protocols for the administration of compositions will be known to one of ordinary skill in the art, in which the dose amount, schedule of injections, sites of injections, mode of administration (e.g., intra-tumoral) and the like vary from the foregoing. Administration of compositions to mammals other than humans, e.g. for testing purposes or veterinary therapeutic purposes, is carried out under substantially the same conditions as described above. A subject, as used herein, is a mammal, preferably a human, and including a non-human primate, cow, horse, pig, sheep, goat, dog, cat or rodent.

When administered, the pharmaceutical preparations of the invention are applied in pharmaceutically-acceptable amounts and in pharmaceutically-acceptable compositions. The term “pharmaceutically acceptable” means a non-toxic material that does not interfere with the effectiveness of the biological activity of the active ingredients. Such preparations may routinely contain salts, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

Compositions may be combined, if desired, with a pharmaceutically-acceptable carrier. The term “pharmaceutically-acceptable carrier” as used herein means one or more compatible solid or liquid fillers, diluents or encapsulating substances which are suitable for administration into a human. The term “carrier” in this context denotes an organic or inorganic ingredient, natural or synthetic, with which the active ingredient is combined to facilitate the application. The components of the pharmaceutical compositions also are capable of being co-mingled with the molecules of the present invention, and with each other, in a manner such that there is no interaction which would substantially impair the desired pharmaceutical efficacy.

The pharmaceutical compositions may contain suitable buffering agents, including: acetic acid in a salt; citric acid in a salt; boric acid in a salt; and phosphoric acid in a salt. The pharmaceutical compositions also may contain, optionally, suitable preservatives, such as: benzalkonium chloride; chlorobutanol; parabens and thimerosal.

The pharmaceutical compositions may conveniently be presented in unit dosage form and may be prepared by any of the methods well-known in the art of pharmacy. All methods include the step of bringing the active agent into association with a carrier which constitutes one or more accessory ingredients. In general, the compositions are prepared by uniformly and intimately bringing the active compound into association with a liquid carrier, a finely divided solid carrier, or both, and then, if necessary, shaping the product.

Compositions suitable for oral administration may be presented as discrete units, such as capsules, tablets, lozenges, each containing a predetermined amount of the active compound. Other compositions include suspensions in aqueous liquids or non-aqueous liquids such as syrup, elixir or an emulsion.

Compositions suitable for parenteral administration conveniently comprise a sterile aqueous or non-aqueous preparation of nucleic acid or immunoglobulin, which is preferably isotonic with the blood of the recipient. This preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono-or di-glycerides. In addition, fatty acids such as oleic acid may be used in the preparation of injectables. Carrier formulation suitable for oral, subcutaneous, intravenous, intramuscular, etc. administrations can be found in Remington's Pharmaceutical Sciences, Mack Publishing Co., Easton, Pa.

In a further preferred embodiment of the invention said pharmaceutical composition further comprises at least one further therapeutic agent; preferably a chemotherapeutic agent.

Preferably said agent is selected from the group consisting of: cisplatin; carboplatin; cyclosphosphamide; melphalan; carmusline; methotrexate; 5-fluorouracil; cytarabine; mercaptopurine; daunorubicin; doxorubicin; epirubicin; vinblastine; vincristine; dactinomycin; mitomycin C; taxol; tamoxifen; L-asparaginase; G-CSF; etoposide; colchicine; derferoxamine mesylate; and camptothecin.

According to a further aspect of the invention there is provided a method to immunise an animal to an antigen, comprising administering an effective amount of an immunoglobulin according to the invention sufficient to stimulate an immune response to at least the first part of said polypeptide.

In an alternative aspect of the invention there is provided a method to immunise an animal to an antigen, comprising administering an effective amount of a nucleic acid or vector according to the invention sufficient to stimulate an immune response to at least the first part of a polypeptide according to the invention.

In a preferred method of the invention said animal is human.

In an alternative preferred method of the invention said animal is selected from the group consisting of: mouse; rat; hamster; goat; cow, horse, pig, dog, cat or sheep.

A preferred route of administration is intradermal, subcutaneous, intramuscular or intranasal, however the immunisation method is not restricted to a particular mode of administration.

According to a further aspect of the invention there is provided a method to produce a hybrid cell-line that produces monoclonal antibodies comprising the steps of:
i) forming a preparation comprising a tumour cell and a hybridoma cell wherein said hybridoma cell is a cell that produces a monoclonal antibody to an immune cell receptor polypeptide;
ii) providing conditions that allow for fusion of said tumour cell and said hybridoma cell and for the proliferation of fused cells; and
iii) screening said fused cells for monoclonal antibodies wherein said antibodies comprise at least two immunoglobulin arms, the first of which binds an immune receptor polypeptide, and the second of which contains the antigen against which an immune response is desired.

In a preferred method of the invention said hybridoma cell is a cell that produces a monoclonal antibody that binds CD40.

In an alternative method of the invention said hybridoma cell is a cell that produces a monoclonal antibody that binds CD28.

In a preferred method of the invention said tumour cell-line is a lymphoma cell-line.

In a preferred method of the invention said tumour cell-line is a primary cell line isolated from a subject that has or is susceptible to cancer. Preferably said cancer is lymphoma.

According to an aspect of the invention there is provided a hybrid cell formed by the method according to the invention.

According to a further aspect of the invention there is provided a cloned population of hybrid cells according to the invention.

According to an aspect of the invention there is provided a monoclonal antibody obtained or obtainable by the method according to the invention.

According to a further aspect of the invention there is provided a method to immunise an animal to an antigen, comprising administering an effective amount of a bi-specific monoclonal antibody according to the invention sufficient to stimulate an immune response to at least one cancer associated antigen.

According to another aspect of the invention there is provided a method to immunise an animal to an antigen, comprising administering an effective amount of a bi-specific monoclonal antibody according to the invention sufficient to stimulate an immune response to at least one pathogen associated antigen

According a yet further aspect of the invention there is provided a method to immunise an animal to an antigen, comprising administering an effective amount of a bi-specific monoclonal antibody according to the invention sufficient to stimulate an immune response to at least one autoimmune disease associated antibody idiotype.

In a preferred method of the invention said animal is human. Preferably said human subjected to immunisation is a human from which said primary tumour cell is isolated.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith

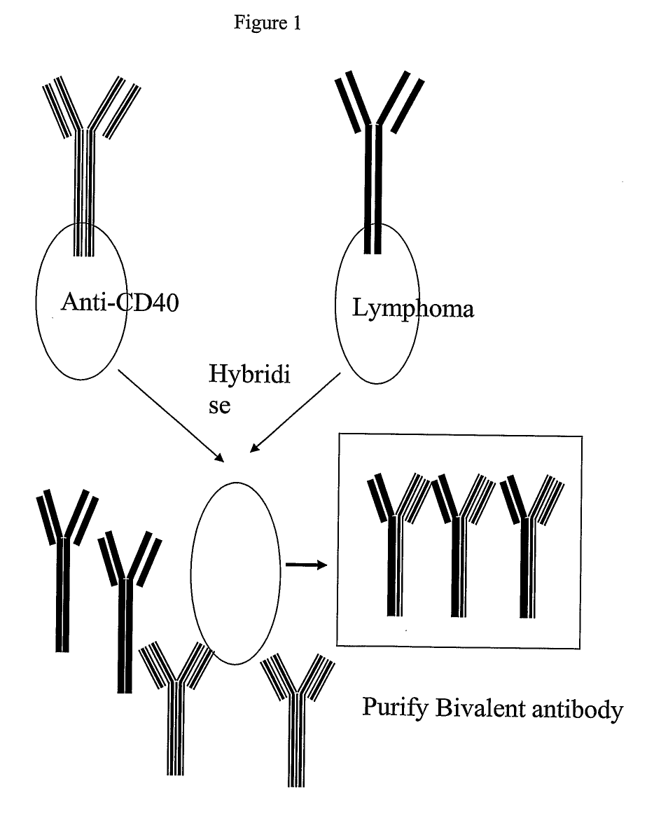

An embodiment of the invention will now be described by example only and with reference to the following methods and FIG. 1:

FIG. 1 illustrates a schematic representation of the formation of hybrid cells formed from a CD40 monoclonal producing cell and a lymphoma cell.

### MATERIALS AND METHODS

### Production of Bi-Valent Antibodies (Lymphoma Idiotype/Anti-CD40)

### Hybridoma Production

Fusions between A20 cells and the rat anti-mouse CD40 hybridoma 10C8 (13) or the control rat IgG1 (anti-human IL12) secreting hybridoma 20C2 (14) are performed using standard fusion techniques (for instance, as described in Hay and Westwood, Practical Immunology, 4^{th} Edition, Blackwell, 2002). Prior to PEG fusion the 10C8 and control 20C2 hybridomas are rendered sensitive to HAT by passage in increasing concentrations of 8-azaguanine (15) while the A20 fusion partner is pretreated prior to fusion with a lethal dose of iodoacetamide (15). Hybrids are selected in HAT containing medium and stable bi-valent antibody producing clones produced by limiting dilution cloning.

In some cases, bivalent antibodies have been produced as a potential tumour therapy, offering targeting to APCs via a specificity for LFA-1 antigen, or CD44 together with an anti-idiotype antibody to target APCs to tumour cells (15, 18). The technical process for production of our Id immunogen is therefore the same, but the end use of the material is quite different, In our case small quantities (10 ug/mouse) of the bivalent antibody are used for active immunisation against the tumour, while for therapy very large, repeated doses are required.

### Bivalent Antibody Purification

Bivalent antibodies for immunisation are purified from supernatants produced by bioreactor growth of the hybrid cell lines immunoglobulin is first purified by Protein G column, and subsequently bivalent antibody is purified by sequential affinity chromatography on anti-rat IgG1, followed by anti-mouse IgG2a affinity columns.

### Screening Assay For Bi-Valent Antibody

The purified bivalent antibody should possess two properties, ability to bind CD40, and the presence of the A20 heavy and light chains. While there is no mAb available against the A20 idiotype, the heavy and light chains can easily be distinguished from the anti-CD40 mAb in being mouse rather than rat derived. Purified immunoglobulin is screened using a sandwich ELISA assay in which recombinant CD40-human Fc is used as capture reagent, and detection is with anti-mouse IgG2a antibody (non rat-reactive). Relative concentrations of control bivalent antibody and CD40 bivalent antibody are assayed using a sandwich ELISA with anti-rat IgG1 to capture and anti-mouse IgG2a, or anti-mouse kappa light chain to detect.

### Immunisations

Mice are immunised one or more times subcutaneously with a low dose (10 ug) of purified bivalent antibody (10C8/A20 or 20C2/A20). Controls are immunised with A20 (Id) antibody alone, or A20 antibody conjugated to KLH.

### Assessment of Anti-Id Responses

### Antibody

Anti-Id antibody responses are assessed by ELISA assay using plates coated with A20 idiotype immunoglobulin in comparison with ELISA using plates coated with purified mouse IgG2a.

### T Cell Proliferation

Proliferation of T cells from vaccinated mice to A20 Id is assessed by a flow cytometric CFSE based assay. The assay allows for the determination of the mean number of cell divisions of both CD4 and CD8 cells (by cell surface staining), as measured by two-fold dilution of the FL1 (CFSE) signal with each cell division. Proliferation in response to A20 Id protein will be assessed in comparison to that induced by a control mouse IgG2a.

### T Cell Cytokine Production

T cell cytokine production in response to A20 Id or control IgG2a protein is assessed by ELISA assay of stimulated cell supernatants, or by intracellular cytokine staining using standard techniques. (Golgi blocking with Brefeldin A, saponin permeabilisation, fixation) Interferon gamma and IL2, or IL4 and IL5 production are assessed as indicative of type 1 or type 2 immune responses respectively.

### CTL Responses

CTL responses are assessed both before and after in vitro re-stimulation with antigen (Id protein, control IgG2a or irradiated A20 cells) by intracellular interferon gamma staining on CD8 cells (see above) or by chromium release assay for killing of A20 lymphoma cells (20). Furthermore CTL activity is directly assessed in vivo by the injection of CFSE labelled A20 cells into immunised or control mice, followed by removal of spleens 18 h later and flow cytometric assessment of A20 cell numbers in the spleen.

### In Vivo Tumour Protection

Mice immunised one or more times as described above are injected subcutaneously with 10⁶ A20 cells⁶. Challenge is normally 30 days post immunisation, but the time from immunisation to challenge may be varied, and therapeutic experiments may also be conducted (challenge before immunisation). Tumour size is monitored by micrometer for 130 days post challenge. In line with UKCCCR guidelines mice with tumours larger than 10% of body weight (15 mm in diameter) are culled. Mean tumour size and survival is compared between groups.

### REFERENCES

1. Stevenson & Stevenson, 1975 Antibody to a molecularly defined antigen confined to a tumour cell surface. Nature 254:714. 2. Vose et al. 2002. A phase 2 trial to evaluate the efficacy of recombinant idiotype vaccine with abbreviated course of granulocyte-macrophage colony-stimulating factor adjuvant in follicular non-Hodgkin's lymphoma. Blood 100; 1397. 3. Stritzke et al 2003 Therapeutic effects of idiotype vaccination can be enhanced by the combination of granulocyte-macrophage colony-stimulating factor and interleukin 2 in a myeloma model. Brit. J. Hematol 120; 27. 4. Bendandil et al. 1999, Nature Med 5:1171. 5. Lundin et al. 2004 CD4(+) T cells kill Id(+) B-lymphoma cells: FasLigand-Fas interaction is dominant in vitro but is redundant in vivo. Cancer Immunol Immunother. 53:1135. 6. Armstrong et al 2002 Immunization with a recombinant adenovirus encoding a lymphoma idiotype: Induction of tumor-protective immunity and identification of an idiotype-specific T cell epitope. J. Immunol, 168:3983. 7. Barr et al. 2003 A potent adjuvant effect of CD40 antibody attached to antigen. Immunology 109:87 8. Alfonso et al. 2002 An anti-idiotype vaccine elicits a specific response to N-glycolyl sialic acid residues of glycoconjugates in melanoma patients. J. Immunol. 168:2523. 9. Foon et al. 2000 Clinical and immune responses in advanced melanoma patients immunized with an anti-idiotype antibody mimicking disialoganglioside GD2 J. Clin Oncol. 18:376. 10. Carlring et al. 2004 CD40 antibody as an adjuvant induces enhanced T cell responses. Vaccine 22; 3323. 11. Chen & Levy 1995 Induction of autoantibody responses to GMCSF by hyperimmunization with an Id-GMCSF fusion protein. J. Immunol. 154:3105. 12. Foy et al. 1993. In-Vivo CD40-gp39 interactions are essential for thymus-dependent humoral immunity. 2. Prolonged suppression of the humoral immune-response by an antibody to the ligand for CD40, Gp39. J Exp Med 178:1567. 13. Barr & Heath. 2001 Functional activity of CD40 antibodies correlates to the position of binding relative to CD154 Immunology 102; 39. 14. Chizzonite et al. 1991 IL-12 monoclonal-antibodies specific for the 40-kda subunit block receptor-binding and biologic activity on activated human lymphoblasts J. Immunol. 147:1548. 15. Kollet et al. 1998 Idiotype-specific inhibition of LFA-1-mediated cell adhesion by anti-idiotype x anti-LFA-1 bispecific antibodies Immunol. Lett. 62; 171. 16. Heath et al. 1990. Monoclonal antibodies mediating viable immunofluorescence and protection against Trypanosoma-cruzi infection Trop. Med. Parasitol 41; 425. 17. Heath et al. 1994 Monoclonal antibodies to murine CD40 define two distinct functional epitopes. Eur. J. Immunol. 24; 1828. 18. Avin et al. 2004 Anti-Idiotype-Anti-CD44 bispecific antibodies inhibit invasion of Lymphoid organs by B Cell lymphoma. J. Immunol 173:4736. 19. Lindhofer et al 1995. Preferential species-restricted heavy/light chain pairing in rat/mouse quadromas: implications for a single-step purification of bispecific Abs. J. Immunol. 155:219. 20. Kronenberger et al. 2002 Impact of the lymphoma idiotype on in vivo tumor protection in a vaccination model based on targeting antigens to antigen-presenting cells. Blood 99:1327

1. A bivalent immunoglobulin polypeptide molecule wherein said molecule comprises a first part that binds to an immune cell receptor polypeptide and a second part that comprises an idiotype to which an immune response is desired.

2. An immunoglobulin according to claim 1 wherein said first part comprises at least one heavy chain or at least one light chain immunoglobulin variable region.

3. An immunoglobulin according to claim 1 wherein said second part comprises at least one immunoglobulin heavy chain or at least one light chain immunoglobulin variable region.

4. A multivalent immunoglobulin polypeptide molecule wherein said polypeptide comprises a first part that includes more than one variable region that binds to an immune cell receptor and a second part that includes more than one idiotope to which an immune response is desired.

5. An immunoglobulin according to claim 1 wherein said immunoglobulin comprises heavy and/or light variable regions selected from the group consisting of: an IgM, an IgD, an IgG, an IgA and an IgE.

6. An immunoglobulin according to claim 1 wherein said immunoglobulin comprises heavy and/or light variable regions of an IgM.

7. An immunoglobulin according to claim 1 wherein said immunoglobulin comprises heavy and/or light variable regions of an IgA.

8. An immunoglobulin according to claim 5 wherein said IgG is selected from the group consisting of: IgG1, IgG2, IgG3 and IgG4.

9. An immunoglobulin according to claim 1 wherein said first or second parts are selected from the group consisting of: an Fᵥ fragment, a Fab fragment, a F(ab′)₂ fragment, a F(ab)₂ fragment, a scFvs fragment, and a single chain antibody fragment, a single domain antibody.

10. An immunoglobulin according to claim 1 wherein said first part consists of a complementarity determining region of an immunoglobulin.

11. An immunoglobulin according to claim 1 wherein said second part comprises at least one idiotope against which an immune response is desired.

12. An immunoglobulin according to claim 1 wherein said first part is crosslinked or conjugated to said second part.

13. An immunoglobulin according to claim 12 wherein said first part and said second part are linked to each other by cross-linking to the same intermediary molecule.

14. An immunoglobulin according to claim 11 wherein said immunoglobulin is associated in a vehicle such as a liposome or a microparticle.

15. An immunoglobulin according to claim 11 wherein said immunoglobulin is co-emulsified in an oil-based emulsion or co-precipitated onto a carrier material.

16. An immunoglobulin according to claim 12 wherein said polypeptide is a fusion protein wherein said first and second parts are in frame translational fusions.

17. An immunoglobulin according to claim 1 wherein said first part binds a member of the tumour necrosis factor receptor superfamily.

18. An immunoglobulin according to claim 17 wherein said first part binds the immune receptor CD40.

19. An immunoglobulin according to claim 1 wherein said first part binds an immune cell receptor selected from the group consisting of: OX40, Fas (CD95), BAFF receptor, BCMA, TACI, APRIL receptor, CD27, CD134, and CD137

20. An immunoglobulin according to claim 1 wherein first part binds a member of the immunoglobulin superfamily other than immunoglobulin.

21. An immunoglobulin according to claim 1 wherein said first part binds the immune receptor CD28.

22. An immunoglobulin according to claim 1 wherein said first part binds an immune receptor selected from the group consisting of: CD152, CD80 or CD86 and ICOS.

23. An immunoglobulin according to claim 1 wherein said first part binds an immune cell receptor selected from the group consisting of: CD154, Fas ligand, APRIL and TRAIL.

24. An immunoglobulin according to claim 1 wherein said first part binds a dendritic cell surface antigen.

25. An immunoglobulin according to claim 1 wherein said first part binds a complement receptor.

26. An immunoglobulin according to claim 1 wherein said first part binds a cytokine receptor or a chemokine receptor.

27. An immunoglobulin according to claim 1 wherein said first part binds a cell adhesion molecule.

28. An immunoglobulin according to claim 1 wherein said immunoglobulin is provided with a sequence tag to facilitate isolation/purification.

29. A nucleic acid molecule comprising a nucleic acid sequence that encodes an immunoglobulin according to claim 16.

30. A vector that comprises a nucleic acid molecule according to claim 29.

31. A vector according to claim 30 wherein said vector is a plasmid, viral based vector, phage or phagemid.

32. A cell transformed or transfected with a nucleic acid or vector according to claim 30.

33. A cell according to claim 32 wherein said cell is a eukaryotic cell.

34. A cell according to claim 32 wherein said cell is a prokaryotic cell.

35. A pharmaceutical composition comprising an immunoglobulin according to claim 1.

36. A pharmaceutical composition comprising a nucleic acid molecule or vector according to claim 29.

37. A composition according to claim 35 wherein said composition further comprises at least one further therapeutic agent.

38. A method to immunise an animal to an antigen, comprising administering an effective amount of an immunoglobulin according to claim 1 sufficient to stimulate an immune response to the immunoglobulin.

39. A method to immunise an animal to an antigen, comprising administering an effective amount of a nucleic acid or vector according to claim 29 sufficient to stimulate an immune response to the immunoglobulin.

40. A method according to claim 38 wherein said animal is human.

41. A method according to claim 38 wherein said animal is selected from the group consisting of: mouse; rat; hamster; goat; cow, horse, pig, dog, cat and sheep.

42. A method to produce a hybrid cell-line that produces monoclonal antibodies comprising the steps of:
i) forming a preparation comprising a tumour cell and a hybridoma cell wherein said hybridoma cell is a cell that produces a monoclonal antibody to an immune cell receptor polypeptide;
ii) providing conditions that allow for fusion of said tumour cell and said hybridoma cell and for the proliferation of fused cells; and
iii) screening said fused cells for monoclonal antibodies wherein said antibodies comprise two arms, the first of which binds an immune receptor polypeptide, and the second of which contains the antigen against which an immune response is desired.

43. A method according to claim 42 wherein said hybridoma cell is a cell that produces a monoclonal antibody that binds CD40.

44. A method according to claim 42 wherein said hybridoma cell is a cell that produces a monoclonal antibody that binds CD28.

45. A method according to claim 42 wherein said tumour cell-line is a lymphoma cell-line.

46. A method according to claim 45 wherein said tumour cell-line is a primary cell line isolated from a subject that has or is susceptible to cancer.

47. A method according to claim 45 wherein said tumour cell line is a fusion between an immortal cell line and a primary tumour cell.

48. A method according to claim 46 wherein said cancer is lymphoma.

49. A hybrid cell formed by the method according to claim 42.

50. A cloned population of hybrid cells according to claim 49

51. A monoclonal antibody obtained or obtainable by the method according to claim 42.

52. A method to immunise an animal to an antigen, comprising administering an effective amount of a bi-specific monoclonal antibody according to claim 51 sufficient to stimulate an immune response to at least one cancer associated antigen.

53. A method to immunise an animal to an antigen, comprising administering an effective amount of a bi-specific monoclonal antibody according to claim 51 sufficient to stimulate an immune response to at least one pathogen associated antigen.

54. A method to immunise an animal to an antigen, comprising administering an effective amount of a bi-specific monoclonal antibody according to claim 51 sufficient to stimulate an immune response to at least one autoimmune disease associated antibody idiotype.

55. A method according to claim 52 wherein said animal is human.

56. A method according to claim 55 wherein said human subjected to immunisation is a human from which said primary tumour cell is isolated.
